(19) United States
(12) Patent Application Publication
(10) Pub. No.: US 2023/0374478 A1
(43) Pub. Date: Nov. 23, 2023

(54) MODIFIED CAS12A PROTEIN AND USE THEREOF

(71) Applicant: G FLAS LIFE SCIENCES, Seoul (KR)

(72) Inventors: Jong Jin PARK, Seoul (KR); Jung Hyuk LEE, Seoul (KR)

(21) Appl. No.: 18/027,598

(22) PCT Filed: Sep. 23, 2021
(86) PCT No.: PCT/KR2021/012936

§ 371 Date: Mar. 21, 2023

(30) Foreign Application Priority Data
Sep. 22, 2020 (KR) 10-2020-0121989
Sep. 22, 2020 (KR) 10-2020-0121990

(51) Int. Cl. C12N 9/22; C12N 15/10

(52) U.S. Cl. CPC C12N 9/22; C12N 15/102; C12N 2310/20; C07K 2319/09

(57) ABSTRACT

The present invention relates to a modified CRISPR-associated protein and a use thereof. More specifically, the present invention relates to a composition for genome editing comprising the modified CRISPR-associated protein and an enhancer, a method for genome editing using the same, and a method for producing a transformant by using the same. The modified CRISPR-associated protein according to the present invention is available as a nuclease having excellent indel efficiency in a CRISPR-Cas system, and exhibits excellent indel efficiency compared to conventional CRISPR-Cas systems, thus finding advantageous applications in genome editing.

### TECHNICAL FIELD

The present invention relates to a modified Cas12a protein and a use thereof. In addition, the present invention relates to a composition for genome editing comprising the modified Cas12a protein and an enhancer, a method for genome editing using the same, and a method for producing a transformant by using the same.

### BACKGROUND ART

Genome editing is a technology that freely corrects the genetic information of living organisms. Advances in the field of life science and developments in genome sequencing technology have enabled us to broadly understand diverse genetic information. For example, although the understanding of genes for animal and plant reproduction, disease and growth, genetic mutations that cause various human genetic diseases, and production of biofuels, and the like has already been secured, further technological advances are essential in order to directly utilize this to improve living organisms and reach the level of treating human diseases.

Genome editing technology can change the genetic information of animals including humans, plants, and microorganisms to dramatically expand its application range. Genetic scissors are molecular tools designed and made to accurately cut the desired genetic information, and play a key role in genome editing technology. Like the next generation sequencing technology that advanced the genetic sequencing field to a new level, genetic scissors are becoming a key technology that expands the speed and scope of genetic information utilization and creates new industrial fields.

The genetic scissors developed so far can be divided into three generations according to the order. ZFN (Zinc Finger Nuclease) is the first-generation genetic scissors, TALEN (Transcription Activator-Like Effector Nuclease) is the second-generation genetic scissors, and CRISPR (Clustered regularly interspaced short palindromic repeat)-Cas (CRISPR-associated) 9, which has been studied most recently, is the third-generation genetic scissors.

CRISPRs are loci comprising multiple short direct repeats found in the genomes of approximately about 40% of bacteria whose genome sequence has been identified and about 90% of archaea whose genome sequence has been identified. When the Cas protein forms a complex with two RNAs, termed CRISPR RNA (crRNA) and trans-activating crRNA (tracrRNA), it forms an active endonuclease, thereby cleaving foreign genetic elements in the invasion of phages or plasmids to protect the host cell. The crRNA is transcribed from the CRISPR element of the host genome that was occupied by the foreign invader upon transfer.

RNA-guided nucleases derived from the CRISPR-Cas system provide a means to edit the genome. In particular, studies related to technology capable of editing the genome of cells and organs using a single guide RNA (sgRNA) and a Cas protein have been actively conducted. Recently, it was reported that the Cpf1 (Cas12a) protein (derived from Prevotella and Francisella 1) is another nuclease protein of the CRISPR-Cas system (B Zetsche, et al, 2015), thus providing a wide range of options for genome editing.

However, the CRISPR-Cas system based on the Cpf1 protein still exhibits the off-target problem of conventional Cas9 protein-based genetic scissors. Non-target DNA cleavage by off-target effects can lead to mutations in undesired genes, such as proto-oncogenes and tumor suppressor genes and can increase genome recombination such as translocation, deletion, and inversion, resulting in a serious problem in using genetic scissors in research fields and medical fields. Therefore, recently, in order to reduce the off-target of genetic scissors, research is being conducted to develop genetic scissors having excellent indel efficiency. However, genetic scissors that operate specifically only at the target site without off-target effects at the whole genome level have not been reported yet.

### DETAILED DESCRIPTION OF INVENTION

### Technical Problem

Accordingly, the present inventors derived a novel CRISPR-associated protein that can be effectively applied to genetic scissors and, based on this, conducted research to develop a genetic scissors system having excellent indel efficiency, thereby completing the present invention.

### Solution to Problem

In order to achieve the above object, in one aspect of the present invention, there is provided a modified Cas12a protein comprising: a Cas12a protein; and myc-NLS (nuclear localization sequences) comprising the amino acid sequence of SEQ ID NO: 4 linked to the C-terminus of the Cas12a protein.

In another aspect of the present invention, there is provided a composition for genome editing comprising: the modified Cas12a protein or a polynucleotide encoding the same; and a guide RNA comprising a nucleotide sequence hybridizable with a target nucleotide sequence, or a polynucleotide encoding the same.

In another aspect of the present invention, there is provided a composition for genome editing comprising: a Cas12a protein, a modified Cas12a protein or a polynucleotide encoding the same; a guide RNA comprising a nucleotide sequence hybridizable with a target nucleotide sequence, or a polynucleotide encoding the same; and an enhancer.

In another aspect of the present invention, there is provided a method for genome editing comprising the step of introducing the composition for genome editing into an isolated cell or an organism.

In another aspect of the present invention, there is provided a method for producing a transformant comprising the step of introducing the composition for genome editing into an isolated cell or an organism except for humans.

### Effects of Invention

The modified Cas12a protein of the present invention has significantly excellent endonuclease activity to recognize and cleave intracellular nucleic acid sequences linked to a guide RNA compared to that of the conventionally used AsCpf1 proteins. Therefore, it can be effectively utilized as a nuclease having excellent indel efficiency in a CRISPR-Cas system. In addition, according to a composition for genome editing comprising a Cas12a protein and an enhancer, and a use thereof, it has excellent indel efficiency compared to that of conventional CRISPR-Cas systems, and thus, it can be effectively utilized for genome editing.

### BRIEF DESCRIPTION OF DRAWINGS

FIG. 1 is a diagram showing the structure of the pET28a-His-opmgCas12a-1-6XNLS-His recombinant expression vector.

FIG. 2 is a photograph identifying the optimal expression conditions of the opmgCas12a-1-6XNLS protein by SDS-PAGE.

FIG. 3 is a graph showing the results of FPLC chromatography (Fast Protein Liquid Chromatography) for identifying opmgCas12a-1-6XNLS using a His column.

FIG. 4 is a graph showing the results of FPLC chromatography for identifying opmgCas12a-1-6XNLS using a desalting column.

FIG. 5 is a photograph showing the results of Coomassie staining for the opmgCas12a-1-6XNLS fraction by SDS-PAGE.

FIG. 6a is a photograph showing a result obtained by comparing knockout efficiencies of AsCas12a, mgCas12a1, opmgCas12a-1-6XNLS, and mgCas12a1-GFP for the HPRT1 gene in HEK293T.

FIG. 6b is a photograph showing a result obtained by comparing knockout efficiencies of AsCas12a, mgCas12a1, opmgCas12a-1-6XNLS, and mgCas12a1-GFP for the HPRT1 gene in HEK293T depending on the presence or absence of an enhancer

FIG. 7a is a graph showing a result obtained by comparing knockout efficiencies of AsCas12a, mgCas12a1, opmgCas12a-1-6XNLS, and mgCas12a1-GFP for the HPRT1 gene in HEK293T.

FIG. 7b is a graph showing a result obtained by comparing knockout efficiencies of AsCas12a, mgCas12a1, opmgCas12a-1-6XNLS, and mgCas12a1-GFP for the HPRT1 gene in HEK293T depending on the presence or absence of an enhancer.

FIG. 8 is a schematic diagram of mgCas12a-1 and mgCas12a-1-6XNLS according to the present invention. Here, BPNLS means bipartite NLS (nuclear localization sequences).

FIG. 9 is a graph showing a result obtained by comparing knockout efficiencies of AsCas12a, mgCas12a-1, and opmgCas12a-1-6XNLS for the HPRT1 gene in HEK293T over time (24 hours and 48 hours) depending on the presence or absence of an enhancer.

### BEST MODE FOR CARRYING OUT THE INVENTION

Modified CRISPR-Associated Cas12a Protein

In one aspect of the present invention, there is provided a modified Cas12a protein comprising: a Cas12a protein; and myc-NLS (nuclear localization sequences) comprising the amino acid sequence of SEQ ID NO: 4 linked to the C-terminus of the protein.

Preferably, the Cas12a protein may comprise the amino acid sequence of SEQ ID NO: 1. In addition, the myc-NLS may comprise the amino acid sequence of SEQ ID NO: 4.

As used herein, the term “Cas12a” is a CRISPR-associated protein and is a type V CRISPR system protein that may also be referred to as Cpf1. Cas12a is a single protein, similar to Cas9, a type II CRISPR system protein, in that it binds to crRNA and cleave a target gene, but differs in its mode of operation. Since the Cas12a protein operates as a single crRNA, there is no need to use crRNA and trans-activating crRNA (tracrRNA) simultaneously, as in the case of Cas9, or artificially construct a single a guide RNA (sgRNA) by combining tracrRNA and crRNA.

In addition, in the Cas12a system, unlike Cas9, the PAM is present at the 5′ position of the target sequence. In addition, the length of the guide RNA (gRNA) that determines the target in the Cas12a system is also shorter than that of Cas9. In addition, since Cas12a generates a 5′ overhang (sticky end) rather than a blunt-end at the position where the target DNA was cleaved, it has the advantage that more accurate and diverse genome editing is possible.

Since mgCas12a has lower indel efficiency than that of AsCpf1 and LbCpf1, it is difficult to apply mgCas12a to the CRISPR-Cas system. However, in the present invention, it was found that mgCas12a-1-6XNLS, in which myc-NLS, a nuclear localization signal, is repeatedly linked to the C-terminus of mgCas12a-1 has significantly excellent indel efficiency. Therefore, it was found that mgCas12a-1-6XNLS may be usefully utilized to improve the genome editing efficiency of the conventional CRISPR-Cas system. In this case, the mgCas12a-1 protein may have the amino acid sequence of SEQ ID NO: 1.

According to one embodiment of the present invention, mgCas12a-1 and myc-NLS may be directly linked. In addition, mgCas12a-1 and myc-NLS may be linked via a linker. The linker may be a peptide linker and may be 1 to 10 amino acids. In addition, the linker may be a peptide composed of 1, 2, 3, 4, 5, 6, 7, 8, 9 or 10 amino acids. Amino acids may be selected from 20 amino acids. Preferably, the peptide linker may comprise amino acids selected from the group consisting of Gly and Ser. In one embodiment, the peptide linker may be Gly-Gly-Ser.

The myc-NLS may be linked to the C-terminus of mgCas12a-1 by the Gly-Gly-Ser amino acid sequence.

In addition, the modified Cas12a protein may further comprise 1 to 10 myc-NLS. In addition, the modified Cas12a protein may comprise six myc-NLS. According to one embodiment of the present invention, six myc-NLS may be repeatedly linked.

According to one embodiment of the present invention, each of the myc-NLS may be linked via a peptide linker. In this case, the peptide linker may be Gly-Gly-Ser.

According to one embodiment, a plurality of myc-NLS linked to the C-terminus of mgCas12a-1 may be repeatedly linked, preferably 2 to 10 myc-NLS may be repeatedly linked, and most preferably six myc-NLS may be repeatedly linked. By repeatedly linking a plurality of myc-NLS to the C-terminus of mgCas12a-1, the genome editing efficiency of mgCas12a-1 may be significantly improved. In addition, myc-NLS may be linked to each other by the Gly-Gly-Ser amino acid sequence.

According to one embodiment of the present invention, the myc-NLS may comprise the amino acid sequence of SEQ ID NO: 4. In addition, the myc-NLS may be encoded by the nucleotide sequence of SEQ ID NO: 3.

According to one embodiment of the present invention, lysine (Lys) at position 169 in the amino acid sequence of SEQ ID NO: 1 may be substituted with arginine (Arg).

According to one embodiment of the present invention, aspartic acid (Asp) at position 529 in the amino acid sequence of SEQ ID NO: 1 may be substituted with arginine (Arg).

In particular, the protein optimized by substituting lysine (Lys) at position 169 in the amino acid sequence of mgCas12a-1-6XNLS of the present invention with arginine (Arg) and substituting aspartic acid (Asp) at position 529 with arginine (Arg) is referred to as opmgCas12a-1-6XNLS. In this case, genome editing efficiency by opmgCas12a-1-6XNLS was significantly increased compared to that of mgCas12a-1-6XNLS.

According to one embodiment of the present invention, the modified Cas12a protein may comprise the amino acid sequence of SEQ ID NO: 22 or SEQ ID NO: 19.

Composition for Genome Editing Comprising Modified Cas12a Protein

In another aspect of the present invention, there is provided a composition for genome editing comprising: the modified Cas12a protein or a polynucleotide encoding the same; and a guide RNA comprising a polynucleotide sequence hybridizable with a target nucleotide sequence, or a polynucleotide encoding the same.

The modified Cas12a protein is as described above.

As used herein, the term “guide RNA (gRNA)” is RNA comprising a nucleotide sequence capable of complementary binding to a target DNA, and gRNA may form a complex with the mgCas12a-1-6XNLS protein, and refers to a single chain RNA capable of bringing the mgCas12a-1-6XNLS protein to the target DNA. In the present invention, a guide RNA may be prepared to be specific to any target to be cleaved.

The modified Cas12a protein of the present invention may be in a form that is easy to be introduced into cells. For example, the modified Cas12a protein may be linked to a cell penetrating peptide or a protein transduction domain. The protein transduction domain may be poly-arginine or HIV-derived TAT protein, but is not limited thereto.

The length of the sequence of the guide RNA capable of forming a base pair with the complementary chain of the target DNA sequence may be 17 to 23 bp, 18 to 23 bp, 19 to 23 bp, more specifically 20 to 23 bp, still more specifically 21 to 23 bp, but is not limited thereto.

The DNA sequence targeted by the guide RNA comprises a PAM (protospacer adjacent motif) sequence of 3 to 4 additional nucleotides upstream of the 5′-terminal region. Specifically, the PAM sequence is preferably 5′-TTTG-3′ or 5′-TTTA-3′.

The modified Cas12a protein, specifically the mgCas12a-1-6XNLS protein, may be linked with a tag advantageous for isolation and/or purification. For example, a small peptide tag such as a His tag, a Flag tag, an S tag, or a GST (glutathione S-transferase) tag, an MBP (maltose binding protein) tag, and the like may be used depending on the purpose, but is not limited thereto. The tag may be linked to the N-terminus or C-terminus of the Cas12a protein or the modified Cas12a protein.

The CRISPR/mgCas12a-1-6XNLS system of the present invention may be used as an isolated polynucleotide such as a polynucleotide encoding a guide RNA; and a polynucleotide encoding a Cas protein. In addition, it may be also used in the form of a recombinant expression vector comprising an expression cassette for expressing a guide RNA or/and a Cas protein.

As used herein, the term “recombinant expression vector” refers to a recombinant DNA molecule comprising a desired coding sequence and an appropriate nucleic acid sequence necessary to express the operably linked coding sequence in a specific host organism. Promoters, enhancers, termination signals and polyadenylation signals available in eukaryotic cells are known.

As used herein, the term “operably linked” refers to a functional linkage between a gene expression control sequence and another nucleotide sequence. The gene expression control sequence may be one or more selected from the group consisting of an origin of replication, a promoter, and a transcription termination sequence (terminator), and the like. The transcription termination sequence may be a polyadenylation sequence (pA), and the origin of replication may be the f1 origin of replication, the SV40 origin of replication, the pMB1 origin of replication, the adeno origin of replication, the AAV origin of replication, or the BBV origin of replication, or the like, but is not limited thereto.

As used herein, the term “promoter” refers to a region of DNA upstream from a structural gene, and refers to a DNA molecule to which RNA polymerase binds in order to initiate transcription.

The promoter according to one embodiment of the present invention is one of the transcription control sequences that control the initiation of transcription of a specific gene, and may be a polynucleotide fragment having a length of about 100 bp to about 2500 bp. The promoter may be used without limitation as long as it may control transcription initiation in cells, for example, eukaryotic cells (for example, plant cells or animal cells (for example, mammalian cells such as humans and mice, etc.), etc.). For example, the promoter may be selected from the group consisting of a CMV promoter (a cytomegalovirus promoter (for example, a human or mouse CMV immediate-early promoter), a U6 promoter, an EF1-alpha (elongation factor 1-a) promoter, an EF1-alpha short (EFS) promoter, an SV40 promoter, an adenovirus promoter (major late promoter), a pL λ promoter, a trp promoter, a lac promoter, a tac promoter, a T7 promoter, a vaccinia virus 7.5K promoter, a tk promoter of HSV, an SV40E1 promoter, a respiratory syncytial virus (RSV) promoter, a metallothionin promoter, a (3-actin promoter, a ubiquitin C promoter, a human IL-2 (human interleukin-2) gene promoter, a human lymphotoxin gene promoter, and a human GM-CSF (human granulocyte-macrophage colony stimulating factor) gene promoter, but is not limited thereto.

The recombinant expression vector according to one embodiment of the present invention may be selected from the group consisting of a plasmid vector, a cosmid vector, a bacteriophage vector, and a viral vector such as an adenoviral vector, a retroviral vector and an adeno-associated viral vector. Vectors that may be used as a recombinant expression vector may be constructed based on plasmids (for example, pcDNA series, pSC101, pGV1106, pACYC177, ColE1, pKT230, pME290, pBR322, pUC8/9, pUC6, pBD9, pHC79, pIJ61, pLAFR1, pHV14, pGEX series, pET series, pUC19, etc.), phages (for example, λgt4λB, λ-Charon, λΔz1, M13, etc.) or viral vectors (for example, an adeno-associated virus (AAV) vector, etc.), etc. used in the art, but is not limited thereto.

The recombinant expression vector of the present invention may further comprise one or more selectable markers. The marker is a nucleic acid sequence having properties that may be selected by conventional chemical methods, and comprises all genes capable of distinguishing transfected cells from non-transfected cells. For example, it may be a herbicide-resistance gene such as glyphosate, glufosinate ammonium or phosphinothricin, an antibiotic-resistance gene such as ampicillin, kanamycin, G418, bleomycin, hygromycin, or chloramphenicol, but is not limited thereto.

The recombinant expression vector of the present invention may be constructed using genetic recombination techniques well known in the art, and site-specific DNA cleavage and linkage may be performed using enzymes generally known in the art, etc.

The composition for genome editing may further comprise an enhancer but is not limited thereto.

As used herein, the term “enhancer” is a part in a gene that induces a structural change in a DNA template and promotes more active transcription, and comprises a unique nucleotide sequence for each gene, and has the properties capable of exerting its function regardless of where it is located in the gene.

The enhancer may be a Cpf1 enhancer, but is not limited thereto. Cpf1 does not require tracrRNA to function, but requires only one short crRNA. In addition, unlike the G-rich PAM of Cas9, Cpf1 recognizes a T-rich PAM (protospacer adjacent motif), allowing new targeting possibilities in the genome. Cpf1 is known to bind to the PAM sequence, 5′-TTN, 5′-TTTN or 5′-TTTV depending on the species of origin. It has been reported that the PAM sequence must be double-stranded for the Cpf1 PAM-binding domain to recognize and bind to the PAM site. Specifically, one specific example of the Cpf1 enhancer may be the oligonucleotide of SEQ ID NO: 9 (TGGATAATAATGAACGCATTAGATAGATTTGAATGCCGGAACTTTGGATTTAGATC ACCCATTGACTTGGTCAACGGAAACATGTTCGCTATTAATATAGCGAACATGTTTC; SEQ ID NO: 24) or SEQ ID NO: 25 (TGGATAATAATGAACGCATTAGATAGATTTGAATGCCGGAACTTTGGATTTAGATC ACCCATTGACTTGGTCAACGGAAACATGTTCGCTATTAATATAGCGAACATGTTTC; SEQ ID NO: 25) disclosed in US Patent Publication No. 2018-0273938. The nucleotide of SEQ ID NO: 9 disclosed in US Patent Publication No. 2018-0273938 has a hairpin length of 16 base pairs and is 46.1% editable. The oligonucleotide of SEQ ID NO: 25 is characterized by having a hairpin length of 16 base pairs and being 44.8% editable.

Composition for Genome Editing Comprising Cas12a Protein and Enhancer

In another aspect of the present invention, there is provided a composition for genome editing comprising: a Cas12a protein or a polynucleotide encoding the same; a guide RNA comprising a nucleotide sequence hybridizable with a target nucleotide sequence, or a polynucleotide encoding the same; and an enhancer.

As used herein, the terms “Cas12a protein”, “guide RNA” and “enhancer” are as described above.

In the present invention, it was found that the type V CRISPR system comprising an enhancer has excellent indel efficiency. In addition, mgCas12a-1 is known to be difficult to apply to the type V CRISPR system since mgCas12a-1 has lower indel efficiency than that of conventional AsCpf1 and LbCpf1. It was found that the type V CRISPR system that uses mgCas12a-1-6XNLS, in which myc-NLS, a nuclear localization signal, is repeatedly linked to the C-terminus of mgCas12a-1, together with an enhancer has significantly excellent indel efficiency. Therefore, it was found that mgCas12a-1-6XNLS as an endonuclease and an enhancer may be usefully utilized to improve the genome editing efficiency of the conventional type V CRISPR system.

The Cas12a protein may have the amino acid sequence of SEQ ID NO: 1. In addition, the Cas12a protein may have a form in which at least one amino acid in SEQ ID NO: 1 is substituted. The substituted form is as described above. In addition, the Cas12a protein may be a modified Cas12a protein. Specifically, as described above, the modified Cas12a protein may be in the form that comprises a protein comprising the amino acid sequence of SEQ ID NO: 1 and myc-NLS.

In addition, the number of myc-NLS to be comprised, linking method, linker, and the like are as described above.

A plurality of myc-NLS linked to the C-terminus of mgCas12a-1 may be repeatedly linked, preferably 2 to 10 myc-NLS may be repeatedly linked, and most preferably six myc-NLS may be repeatedly linked. By repeatedly linking a plurality of myc-NLS to the C-terminus of mgCas12a-1, the genome editing efficiency of mgCas12a-1 may be significantly improved. In addition, myc-NLS may be linked to each other by the Gly-Gly-Ser amino acid sequence.

According to one embodiment of the present invention, the myc-NLS may comprise the nucleotide sequence of SEQ ID NO: 3. In addition, the myc-NLS may comprise the amino acid sequence of SEQ ID NO: 4.

In particular, effective genome editing may be achieved by opmgCas12a-1-6XNLS optimized by substituting lysine (Lys) at position 169 with arginine (Arg) and substituting aspartic acid (Asp) at position 529 with arginine (Arg) in the amino acid sequence of mgCas12a-1-6XNLS of the present invention.

According to one embodiment of the present invention, the Cas12a protein may comprise the amino acid sequence of SEQ ID NO: 22 or SEQ ID NO: 19.

Use of Composition for Genome Editing Comprising Cas12a Protein and/or Enhancer

In another aspect of the present invention, there is provided a method for genome editing comprising introducing the composition for genome editing into an isolated cell or an organism.

In another aspect of the present invention, there is provided a method for producing a transformant comprising the step of introducing a composition for genome editing into an isolated cell or an organism except for humans.

The composition for genome editing of the present invention may be introduced into the cell or the organism by methods known in the art for introducing nucleic acid molecules into the organism, cell, tissue or organ, and as known in the art, may be performed with a selection of standard techniques suitable for host cells. These methods may comprise, for example, electroporation, calcium phosphate (CaPO₄) precipitation, calcium chloride (CaCl₂) precipitation, microinjection, polyethylene glycol (PEG) method, DEAE-dextran method, cationic liposome method, and lithium acetate-DMSO method, and the like, but are not limited thereto.

### MODE FOR CARRYING OUT THE INVENTION

Hereinafter, the present invention will be described in more detail through one or more examples. However, these examples are intended to illustrate the present invention by way of example, and the scope of the present invention is not limited to these Examples.

### Example 1. Construction of mgCas12a-1 Mutant Recombinant Vector Having Increased Genome Editing Efficiency

### Example 1.1. pET28a-his-mgCas12a-1-6XNLS-his Recombinant Vector

In order to increase the genome editing efficiency of the protein (mgCas12a-1) having the amino acid sequence of SEQ ID NO: 1 encoded by the nucleotide sequence of SEQ ID NO: 2, the protein mutant (mgCas12a-1-6XNLS) recombinant vector was constructed by adding myc-NLS (nuclear localization sequences) of Table 1 to the C-terminus of mgCas12a-1 repeatedly 6 times.

TABLE 1

| Name | Item | Sequence | SEQ ID NO |
|---|---|---|---|
| myc-NLS | nucleotide | ccggcagcca | SEQ ID NO: 3 |
| | sequence | agagagtcaa | |
| | (5′→3′) | actcgac | |
| | amino acid | PAAKRVKLD | SEQ ID NO: 4 |
| | sequence | | |
| | (N→C) | | |

Specifically, inserts were prepared by ordering and annealing the core region (130 bp) of Table 2 in the form of primers, and then PCR amplification using the extension primers of Table 3.

TABLE 2

| Name | Nucleotide sequence (5′→3′) | SEQ ID NO |
|---|---|---|
| Core | agccaagagagtcaaactcgacggcgg | SEQ ID |
| region | ctccccagcggcaaaaagggtgaaact | NO: 5 |
| | agacgggggtagccccgccgcgaagcg | |
| | tgtaaagctggatggaggatcgcctgc | |
| | ggctaagcgagtcaaattagac | |

TABLE 3

| | | Primer nucleotide | SEQ ID |
|---|---|---|---|
| Primer | | sequence (5′→3′) | NO |
| extension | forward | ttgacttcattcaaaa | SEQ ID |
| | | taagcggtatctgggc | NO: 6 |
| | | ggctcccctgctgcta | |
| | | aacgtgttaagcttga | |
| | | tgggggtagcccggca | |
| | | gccaagagagtcaaac | |
| | | tcg | |
| primer | reverse | agccggatctcagtgg | SEQ ID |
| | | tggtggtggtggtggc | NO: 7 |
| | | tgccgctagcatccaa | |
| | | tttgacgcgctttgca | |
| | | gccggtgacccaccgt | |
| | | ctaatttgactcgctt | |
| | | agcc | |

On the other hand, a vector was prepared by double-cutting the pET28a-6XHis-mgCas12a-1 plasmid, which is conventionally used, by NotI and SalI. Thereafter, the insert was cloned into the vector by performing Gibson assembly. As a result, it was found that the pET28a-His-mgCas12a-1-6XNLS-His structure was constructed in three colonies. The mgCas12a-1-6XNLS protein has the amino acid sequence of SEQ ID NO: 8 encoded by the nucleotide sequence of SEQ ID NO: 9.

### Example 1.2. pET28a-his-opmgCas12a-1-6XNLS-his Recombinant Vector

In the mgCas12a-1-6XNLS recombinant vector of Example 1.1., the opmgCas12a-1-6XNLS recombinant vector, which was codon optimized by point mutation, was constructed.

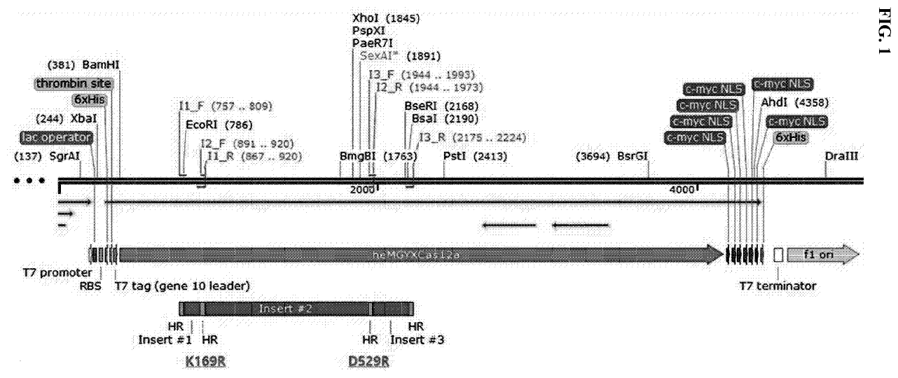

Specifically, it was designed to generate point mutations (K169R and D529R) at homologous recombination (HR) sites between inserts (FIG. 1). Then, after PCR amplification, DpnI was treated, and PCR products were obtained to prepare three inserts, respectively.

TABLE 4

| Nucleotide sequence | | Forward | Reverse |
|---|---|---|---|
| of Inserts (5′→3′) | | primer | primer |
| 1 | SEQ ID NO: 10 | SEQ ID NO: 11 | SEQ ID NO: 12 |
| | gcgccaaattgatta | gcgccaaattgat | gtcggcgctaaag |
| | gtgacatcctgccgg | tagtgacatcctg | caatttgcgcggt |
| | aattcgttattcaca | ccggaattcgtta | tgcggaaataatc |
| | ataacaattactctg | ttcacaataacaa | tttaaatgaagtg |
| | ctagcgagaaggaag | tt | gc |
| | agaaaacccaagtca | | |
| | taaagctcttttccc | | |
| | ggttcgccacttcat | | |
| | ttaaagattatttcc | | |
| | gcaaccgcgcaaatt | | |
| | gctttagcgccgac | | |
| 2 | SEQ ID NO: 13 | SEQ ID NO: 14 | SEQ ID NO: 15 |
| | cgcaaccgcgcaaat | cgcaaccgcgcaa | gcgggccagagtc |
| | tgctttagcgccgac | attgctttagcgc | ggaatgccgaagt |
| | gatatcagttctagc | cgac | tcaa |
| | tcctgtcatcggatt | | |
| | gtgaacgacaatgct | | |
| | gaaatcttcttttca | | |
| | aacgcccttgtatac | | |
| | cgccggattgtgaaa | | |
| | aatctgagcaacgat | | |
| | gacataaataagatc | | |
| | agtggagatattaaa | | |
| | gactctttgaaggag | | |
| | atgagcctggaagag | | |
| | atctattcctacgaa | | |
| | aaatatggggagttc | | |
| | attacccaggaaggc | | |
| | atatcattttacaac | | |
| | gatatctgcggtaag | | |
| | gttaatagcttcatg | | |
| | aacctctattgtcag | | |
| | aaaaataaggagaac | | |
| | aaaaatctttacaag | | |
| | ctgcgcaaattgcac | | |
| | aagcaaattctgtgc | | |
| | atcgcagacacaagt | | |
| | tatgaagtcccttac | | |
| | aaatttgagtctgat | | |
| | gaagaggtgtatcag | | |
| | agcgtaaacggcttc | | |
| | ctcgacaatatttcc | | |
| | tcaaagcatatagtg | | |
| | gaacggcttcgcaaa | | |
| | atcggagataactac | | |
| | aatgggtataacctg | | |
| | gacaagatttacatc | | |
| | gttagcaaattttat | | |
| | gagagtgtctctcag | | |
| | aagacctaccgggat | | |
| | tgggaaactattaat | | |
| | accgccttggagata | | |
| | cactataacaatatc | | |
| | ctgcccggcaacggt | | |
| | aaaagcaaggctgac | | |
| | aaagtgaagaaagcc | | |
| | gtaaagaatgatctc | | |
| | caaaaatccattaca | | |
| | gaaatcaacgagctt | | |
| | gtgtcaaattacaag | | |
| | ctgtgtccggacgat | | |
| | aacattaaagcagaa | | |
| | acctatatacatgag | | |
| | atcagccacattttg | | |
| | aataacttcgaagcc | | |
| | caggagctgaagtac | | |
| | aatccagaaatccat | | |
| | ctcgttgagagtgaa | | |
| | cttaaagcttctgag | | |
| | ctgaagaacgtcttg | | |
| | gacgtgattatgaat | | |
| | gcctttcactggtgc | | |
| | agcgtattcatgact | | |
| | gaagagctggtggat | | |
| | aaagacaacaatttt | | |
| | tatgcagaactcgag | | |
| | gaaatatacgatgag | | |
| | atctataccgttatt | | |
| | tccctttacaacctg | | |
| | gtccgcaattatgtg | | |
| | acacagaagccctac | | |
| | tcaaccaaaaagatc | | |
| | aaattgaacttcggc | | |
| | attccgactctggcc | | |
| | cgc | | |
| 3 | SEQ ID NO: 16 | SEQ ID NO: 17 | SEQ ID NO: 18 |
| | ataacgctataatcc | ttgaacttcggca | agaatatatgcgc |
| | tcatgcgggataatc | Ltccgactctggc | tgggcttgtaggt |
| | tttactatctgggga | ccgcggatggagc | ctccactcctgtt |
| | tttttaacgccaaga | aagagtaaaga | tttgaggagag |
| | ataaacctgacaaga | | |
| | aaatcattgagggca | | |
| | acaccagcgaaaata | | |
| | agggtgattacaaaa | | |
| | agatgatatataact | | |
| | tgctgcccggcccga | | |
| | ataaaatgatcccaa | | |
| | aggtattcctctcct | | |
| | caaaaacaggagtgg | | |
| | agacctacaagccca | | |
| | gcgcatatattct | | |

On the other hand, a vector was prepared by double-cutting the pET28a-His-mgCas12a-1-6XNLS-His vector of Example 1.1. using EcoRI and BsaI. Thereafter, the insert was cloned into the vector by performing Gibson assembly to construct the pET28a-His-opmgCas12a-1-6XNLS-His recombinant vector in which opmgCas12a-1 was cloned. The opmgCas12a-1-6XNLS-His protein has the amino acid sequence of SEQ ID NO: 19 encoded by the nucleotide sequence of SEQ ID NO: 20.

### Example 2. Identification of Optimal Conditions for opmgCas12a-1-6XNLS Protein Expression

### Example 2.1. Transformation

The opmgCas12a-1-6XNLS expression recombinant vector constructed in Example 1.2. was transformed into competent cells, Rosetta(DE3) or Rosetta2(DE3) pLysS, respectively.

Specifically, 1 μL of the recombinant vector of Example 1.2. was added to 100 μL of Rosetta(DE3) or Rosetta2(DE3) pLysS and then was reacted on ice for 30 minutes. Thereafter, heat shock was applied at 42° C. for 45 seconds, and then it was quickly transferred to ice and reacted for 2 minutes. 1 mL of LB medium was added, and then incubation was performed with shaking at 37° C. for 1 hour, and then the cells were precipitated by centrifugation at 13,000 rpm for 3 minutes, and 100 μL of the supernatant was collected and then resuspended, and it was spread on an LB plate containing kanamycin, and then incubated at 37° C. overnight to transform it.

### Example 2.2. Identification of Protein Expression Level

In order to identify the expression level of the opmgCas12a-1-6XNLS protein, the optimal conditions for expression of Rosetta(DE3) and Rosetta2(DE3) pLysS transformed in Example 2.1. were analyzed.

Specifically, Rosetta(DE3) or Rosetta2(DE3) pLysS transformed in Example 2.1. was incubated overnight, and 5 mL was inoculated into 500 mL of liquid TB medium supplemented with 100 mg/ml of kanamycin antibiotic and then incubated in a 37° C. incubator until an OD₆₀₀ reached 0.6. The expression of the opmgCas12a-1-6XNLS protein was induced by treatment with 0.5 mM IPTG (isopropyl β-D-1-thiogalactopyranoside), and then incubation was performed at 18° C. or 28° C. for 18 hours. The cells obtained after centrifugation were mixed with 10 mL of lysis buffer (20 mM HEPES pH 7.5, 100 mM KCl, 20 mM imidazole, 10% glycerol and EDTA-free protease inhibitor cocktail), and then the cells were disrupted by ultrasonic treatment. The disrupted product was centrifuged 3 times for 20 minutes at 6,000 rpm and then filtered through a 0.22 micron filter. Thereafter, it was washed and eluted with a nickel column (HisTrap FF 5 mL) and 300 mM imidazole buffer, and then SDS-PAGE electrophoresis and coomassie staining were performed to identify the opmgCas12a-1-6XNLS protein.

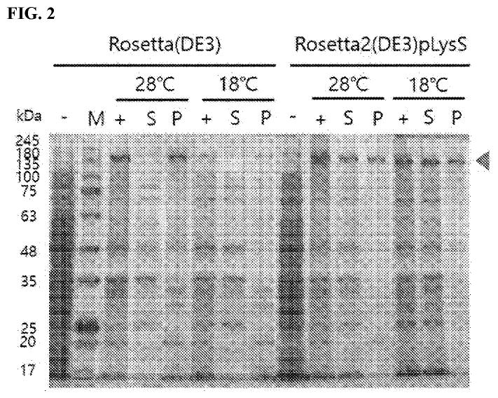

As a result, it was found that the incubation condition at 18° C. for 18 hours using Rosetta2(DE3) pLysS was an optimal condition for the expression of the opmgCas12a-1-6XNLS protein (FIG. 2).

### Example 3. Purification of opmgCas12a-1-6XNLS Protein

The opmgCas12a-1-6XNLS protein washed and eluted by a method of Example 2 was purified by chromatography.

Specifically, the opmgCas12a-1-6XNLS protein was purified by FPLC chromatography using a His column or a desalting column. It was dialyzed overnight with dialysis buffer (20 mM HEPES pH 7.5, 100 mM KCl, 1 mM DTT, 10% glycerol), and selectively filtered and concentrated depending on protein size (Amicon Ultra Centrifugal Filter 100,000 MWCO) to purify the opmgCas12a-1-6XNLS protein.

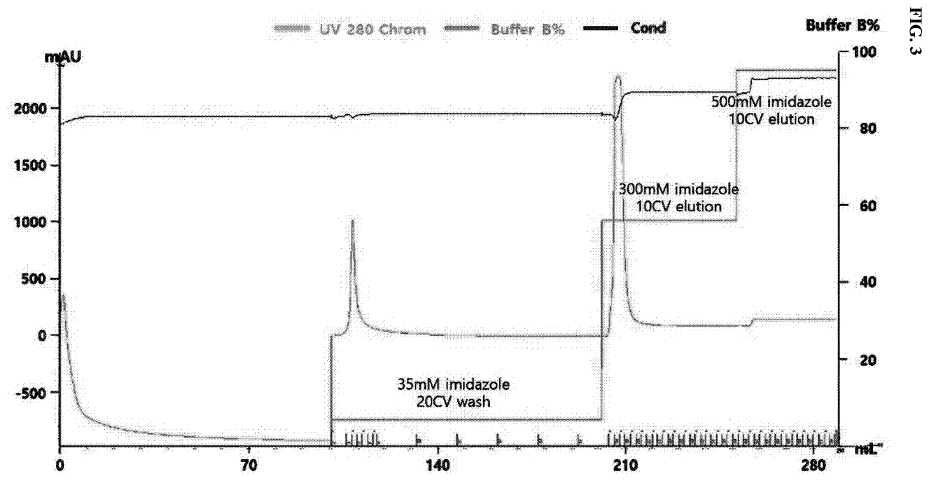

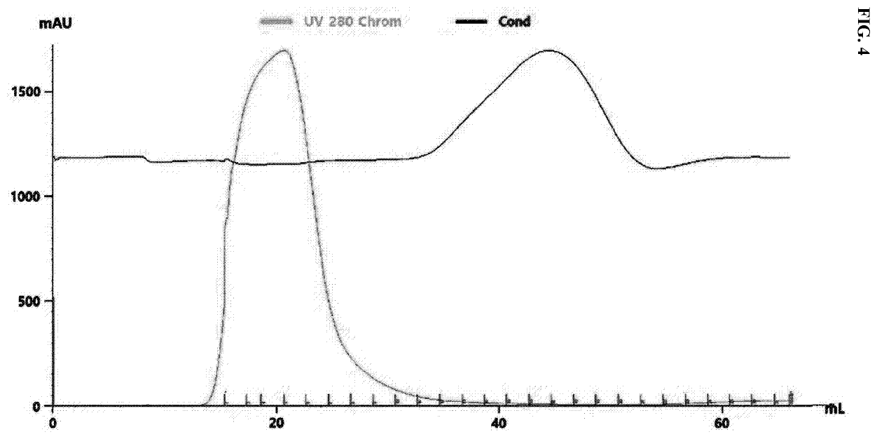

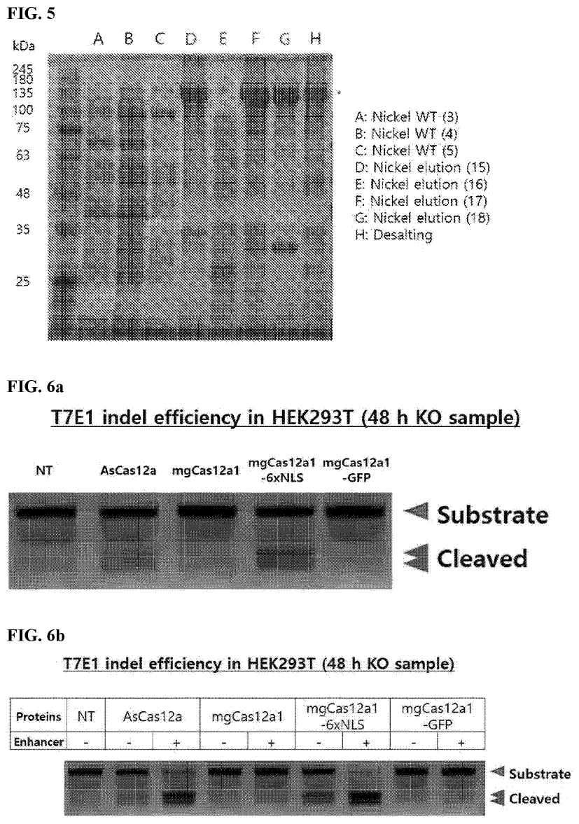

SDS-PAGE electrophoresis and coomassie staining were performed to identify the purification yield of the opmgCas12a-1-6XNLS protein. As a result, it was found that the protein concentration was 35.54 mg/ml, the protein volume was 1.6 mL, and the purification yield of the opmgCas12a-1-6XNLS protein was 56.9 mg/L (FIGS. 3 to 5).

Thereafter, the concentration of the purified opmgCas12a-1-6XNLS protein was measured by the Bradford quantification method, and it was stored at −80° C. before use.

### Example 4. Identification of Enhanced Genome Editing Efficiency of opmgCas12a-1-6XNLS

### Example 4.1. Construction of RNP comprising opmgCas12a-1-6XNLS for HPRT1 genome editing

HEK 293T cells were incubated in DMEM (Dulbecco Modified Eagle Medium) containing 10% fetal bovine serum FBS and P/S (penicillin-streptomycin) at 37° C. in a 5% CO₂ incubator. The ribonucleic acid protein (RNP) was constructed by incubating 126 pmol of the opmgCas12a-1-6XNLS protein 5 μM and 160 pmol of crRNA (Table 4) 6.4 μM targeting HPRT1 at room temperature for 20 minutes. The crRNA sequence of HPRT1 used was synthesized from IDT (Integrated DNA Technologies).

TABLE 5

| | crRNA sequence | SEQ ID |
|---|---|---|
| Gene | (5′->3′) | NO |
| HPRT1 | GGTTAAAGATG | SEQ ID |
| | GTTAAATGAT | NO: 21 |

2×10⁵ of HEK293T cells were mixed with 20 μL of nucleofection reagent and mixed with 10 μL of RNP, or 10 μL of RNP and enhancer (final concentration of 3 μM), and then RNP was introduced into cells by electroporation using a 4D-Nucleofector instruments (Lonza). As an enhancer, IDT's ‘Cpf1 electroporation enhancer, 10 nmol (cat no. 1076301)’ was purchased and used. After 48 hours of transformation, genomic DNA was extracted from the cells using the PURELINK™ Genomic DNA Mini Kit (Invitrogen).

### Example 4.2. Sequencing Analysis for Target Site

The genomic DNA extracted in Example 4.1. was purified according to the protocol of Illumina, and a sequencing library was prepared, and deep sequencing analysis was performed on the target site using MiniSeq equipment.

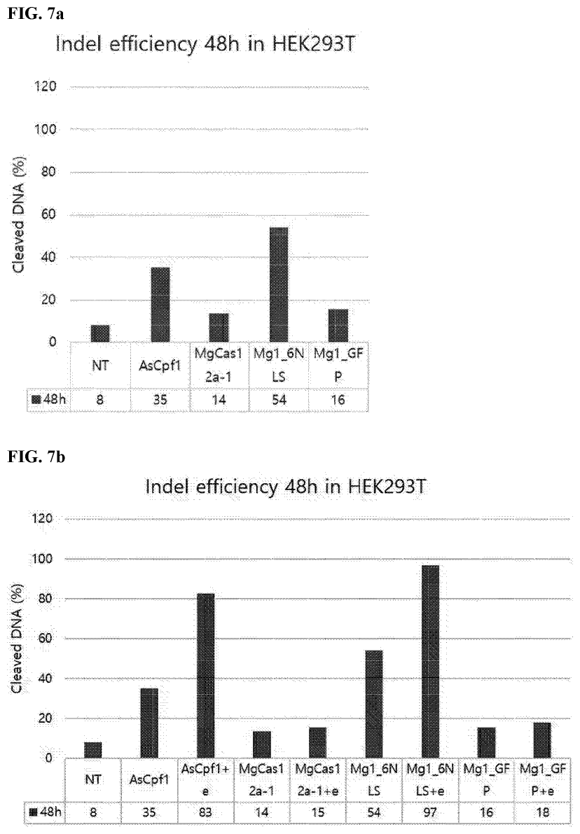

As a result, the knockout efficiency of the HPRT1 gene of opmgCas12a-1-6XNLS was found to be excellent by about 4 times compared to that of mgCas12a-1, and the knockout efficiency was found to be excellent by about 2 times compared to that of the commonly used AsCpf1. Therefore, it was found that the opmgCas12a-1-6XNLS protein had excellent genome editing efficiency (FIGS. 6a and 7b). In addition, when an enhancer was used, knockout efficiency was found to be close to 100%. Accordingly, it was found that the genome editing efficiency by the opmgCas12a-1-6XNLS protein and the enhancer was excellent (FIGS. 6b and 7b).

### Example 5. Identification of Enhanced Genome Editing Efficiency of opmgCas12a-1-6XNLS Over Time

An RNP comprising opmgCas12a-1-6XNLS (FIG. 8) for HPRT1 genome editing was constructed in the same manner as in Example 4.1. above, and the nucleotide sequence for the target site was analyzed in the same manner as in Example 4.2.

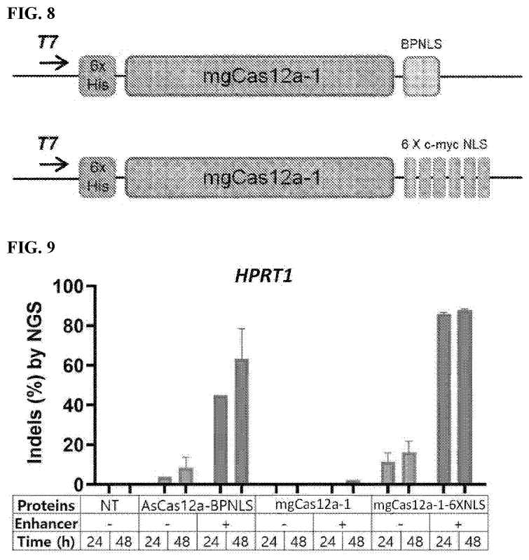

As a result of identifying the genome editing efficiency over time, it was found that the knockout efficiency of the HPRT1 gene of opmgCas12a-1-6XNLS was significantly excellent compared to that of mgCas12a-1, and similarly, the knockout efficiency was excellent compared to that of the commonly used AsCpf1. In addition, knockout efficiency was found to be close to 100% even after 24 hours when an enhancer was used. Accordingly, it was found that the genome editing efficiency by the opmgCas12a-1-6XNLS protein and the enhancer was excellent (FIG. 9).

1. A modified Cas12a protein comprising:
a Cas12a protein; and
myc-NLS (nuclear localization sequences) comprising the amino acid sequence of SEQ ID NO: 4 linked to the C-terminus of the protein.

2. The modified Cas12a protein according to claim 1, wherein the Cas12a protein comprises the amino acid sequence of SEQ ID NO: 1.

3. The modified Cas12a protein according to claim 1, wherein the linkage is by a peptide linker.

4. The modified Cas12a protein according to claim 1, wherein the modified Cas12a protein further comprises 2 to 10 myc-NLS.

5. The modified Cas12a protein according to claim 4, wherein the modified Cas12a protein further comprises six myc-NLS.

6. The modified Cas12a protein according to claim 4, wherein each of the myc-NLS is linked by the Gly-Gly-Ser amino acid sequence.

7. The modified Cas12a protein according to claim 1, wherein lysine (Lys) at position 169 in the amino acid sequence of SEQ ID NO: 1 is substituted with arginine (Arg).

8. The modified Cas12a protein according to claim 1, wherein aspartic acid (Asp) is substituted with arginine (Arg) at position 529 in the amino acid sequence of SEQ ID NO: 1.

9. The modified Cas12a protein according to claim 1, wherein the Cas12a protein comprises the amino acid sequence of SEQ ID NO: 22.

10. A composition for genome editing comprising:
the modified Cas12a protein according to claim 1 or a polynucleotide encoding the same; and
a guide RNA comprising a nucleotide sequence hybridizable with a target nucleotide sequence, or a polynucleotide encoding the same.

11. The composition for genome editing according to claim 10, wherein the composition for genome editing further comprises an enhancer.

12. A composition for genome editing comprising:
a Cas12a protein or a polynucleotide encoding the same;
a guide RNA comprising a nucleotide sequence hybridizable with a target nucleotide sequence, or a polynucleotide encoding the same; and
an enhancer.

13. The composition for genome editing according to claim 12, wherein the Cas12a protein is a modified Cas12a protein comprising:
a protein comprising the amino acid sequence of SEQ ID NO: 1; and
myc-NLS (nuclear localization sequences) comprising the amino acid sequence of SEQ ID NO: 4 linked to the C-terminus of the protein.

14. A method for genome editing comprising introducing the composition for genome editing according to claim 10 into an isolated cell or an organism.

15. A method for producing a transformant comprising the step of introducing the composition for genome editing according to claim 10 into an isolated cell or an organism except for humans.


SEQUENCE LISTING (25 sequences)

SEQ ID NO: 1 - protein, 1330 aa, Artificial Sequence
  mgCas12a-1
MGSSHHHHHHSSGLVPRGSHMASMTGGQQMGRGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLAAALEKRPAATKKAGQAKKKKSTPPPPPLRSGC

SEQ ID NO: 2 - DNA, 3993 bp, Artificial Sequence
  mgCas12a-1
atgggcagcagccatcatcatcatcatcacagcagcggcctggtgccgcgcggcagccatatggctagcatgactggtggacagcaaatgggtcgcggatccatgaacaatggcaccaacaatttccagaactttatcggaattagcagtctgcaaaagactctccggaatgcccttatacccaccgagacaacccagcagttcatcgtgaaaaacgggattatcaaggaagacgagctgcgcggcgaaaatcggcaaattttgaaagatataatggacgattattaccgcggttttatctctgagactctgagctccattgacgatatcgactggacctcactcttcgaaaagatggagattcagcttaaaaacggcgataataaggacacactgataaaagaacaggctgagaagcggaaagccatctataagaaatttgcagatgacgatcgcttcaagaacatgtttagcgccaaattgattagtgacatcctgccggaattcgttattcacaataacaattactctgctagcgagaaggaagagaaaacccaagtcataaagctcttttcccggttcgccacttcatttaaagattatttcaagaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggccgacggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattcttgaagggtacaaacaaaacaagcatctgaaaagttctaaggactttgatatcactttctgtcacgacttgattgattattttaaaaattgcatagccatccatccggagtggaagaacttcggctttgacttcagcgatacctccacatacgaagacatttcaggtttttatcgcgaggttgaactgcagggctacaaaatcgattggacctatattagcgagaaggacatagatctccttcaggaaaaaggacaactgtacttgttccagatctataataaggactttagtaaaaagtctactgggaacgataatctgcacaccatgtacctcaaaaaccttttcagcgaggaaaatctgaaggacattgtcttgaaactgaacggcgaggctgaaatctttttccggaagtcctcaattaaaaatcctataatccataagaaaggtagcattctcgtgaaccgcacatatgaggccgaagagaaggatcagtttggcaatatccaaattgtacggaaaaccatacccgaaaacatctaccaggagctttataagtacttcaatgacaaaagtgataaggaactgtctgacgaggcagccaaattgaagaacgtggttggacaccatgaagctgccactaatattgtcaaagattatcgctacacctatgacaagtactttctgcacatgccgatcacaattaacttcaaagcaaataagaccagctttataaacgatcggattctccagtatattgccaaagagaagaatcttcatgtgatcgggattgaccgcggcgaacggaacctgatatacgtatccgtgatcgatacttgtggtaatattgttgagcaaaaatcattcaacatcgtcaatggctatgactaccagattaagttgaaacagcaagaaggagctcgccagatagcccggaaggagtggaaggaaatcgggaaaattaaggagatcaaagaaggctatctgagcctcgtgattcacgagataagtaagatggtaatcaaatacaacgcaattatcgccatggaagatctttcttatggttttaagaaaggccgcttcaaggtggagcggcaagtttaccagaaatttgaaaccatgctgattaataagttgaactatctggtcttcaaagacataagcatcacagagaatggagggctccttaagggctaccagctgacctatattccagataaattgaagaacgtgggtcatcaatgcggctgtatcttttacgtacccgctgcctatacttccaaaattgacccgaccacaggattcgtgaatatatttaagttcaaagatctgaccgttgacgcaaagcgcgaatttatcaaaaagttcgattcaattcggtacgacagcgagaaaaagctcttttgcttcacttttgattataacaatttcatcacccagaacacagtcatgagtaaatctagctggtccgtgtacacctatggggtacgcattaagcggcgctttgtgaatggccggttctcaaacgaaagcgacactatagatatcaccaaagacatggagaagacacttgaaatgaccgatattaattggcgcgacggtcacgatctgcggcaggacatcattgattacgagatagttcaacatatctttgaaattttccgcttgactgtccagatgcggaacagtctgtctgagctcgaagaccgcgattatgaccggcttatcagccctgtgctgaatgagaacaatattttttacgattccgccaaagctggcgacgccttgcccaaggatgcagacgccaacggagcttattgtatagccctgaaagggctctacgaaatcaagcagattaccgagaattggaaagaagatggcaagttctcacgcgacaaacttaagatcagcaacaaagattggtttgacttcattcaaaataagcggtatctggcggccgcactcgagaaaaggccggcggccacgaaaaaggccggccaggcaaaaaagaaaaagtcgacaccaccaccaccaccactgagatccggctgctaa

SEQ ID NO: 3 - DNA, 27 bp, Artificial Sequence
  myc-NLS
ccggcagccaagagagtcaaactcgac

SEQ ID NO: 4 - protein, 9 aa, Artificial Sequence
  myc-NLS
PAAKRVKLD

SEQ ID NO: 5 - DNA, 130 bp, Artificial Sequence
  Core region
agccaagagagtcaaactcgacggcggctccccagcggcaaaaagggtgaaactagacgggggtagccccgccgcgaagcgtgtaaagctggatggaggatcgcctgcggctaagcgagtcaaattagac

SEQ ID NO: 6 - DNA, 99 bp, Artificial Sequence
  extension primer_forward
ttgacttcattcaaaataagcggtatctgggcggctcccctgctgctaaacgtgttaagcttgatgggggtagcccggcagccaagagagtcaaactcg

SEQ ID NO: 7 - DNA, 100 bp, Artificial Sequence
  extension primer_reverse
agccggatctcagtggtggtggtggtggtggctgccgctagcatccaatttgacgcgctttgcagccggtgacccaccgtctaatttgactcgcttagcc

SEQ ID NO: 8 - protein, 1379 aa, Artificial Sequence
  mgCas12a-1-6XNLS
MGSSHHHHHHSSGLVPRGSHMASMTGGQQMGRGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGSPAAKRVKLDGGSPAAKRVKLDGGSPAAKRVKLDGGSPAAKRVKLDGGSPAAKRVKLDGGSPAAKRVKLDASGSHHHHHH

SEQ ID NO: 9 - DNA, 4140 bp, Artificial Sequence
  mgCas12a-1-6XNLS
atgggcagcagccatcatcatcatcatcacagcagcggcctggtgccgcgcggcagccatatggctagcatgactggtggacagcaaatgggtcgcggatccatgaacaatggcaccaacaatttccagaactttatcggaattagcagtctgcaaaagactctccggaatgcccttatacccaccgagacaacccagcagttcatcgtgaaaaacgggattatcaaggaagacgagctgcgcggcgaaaatcggcaaattttgaaagatataatggacgattattaccgcggttttatctctgagactctgagctccattgacgatatcgactggacctcactcttcgaaaagatggagattcagcttaaaaacggcgataataaggacacactgataaaagaacaggctgagaagcggaaagccatctataagaaatttgcagatgacgatcgcttcaagaacatgtttagcgccaaattgattagtgacatcctgccggaattcgttattcacaataacaattactctgctagcgagaaggaagagaaaacccaagtcataaagctcttttcccggttcgccacttcatttaaagattatttcaagaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggccgacggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattcttgaagggtacaaacaaaacaagcatctgaaaagttctaaggactttgatatcactttctgtcacgacttgattgattattttaaaaattgcatagccatccatccggagtggaagaacttcggctttgacttcagcgatacctccacatacgaagacatttcaggtttttatcgcgaggttgaactgcagggctacaaaatcgattggacctatattagcgagaaggacatagatctccttcaggaaaaaggacaactgtacttgttccagatctataataaggactttagtaaaaagtctactgggaacgataatctgcacaccatgtacctcaaaaaccttttcagcgaggaaaatctgaaggacattgtcttgaaactgaacggcgaggctgaaatctttttccggaagtcctcaattaaaaatcctataatccataagaaaggtagcattctcgtgaaccgcacatatgaggccgaagagaaggatcagtttggcaatatccaaattgtacggaaaaccatacccgaaaacatctaccaggagctttataagtacttcaatgacaaaagtgataaggaactgtctgacgaggcagccaaattgaagaacgtggttggacaccatgaagctgccactaatattgtcaaagattatcgctacacctatgacaagtactttctgcacatgccgatcacaattaacttcaaagcaaataagaccagctttataaacgatcggattctccagtatattgccaaagagaagaatcttcatgtgatcgggattgaccgcggcgaacggaacctgatatacgtatccgtgatcgatacttgtggtaatattgttgagcaaaaatcattcaacatcgtcaatggctatgactaccagattaagttgaaacagcaagaaggagctcgccagatagcccggaaggagtggaaggaaatcgggaaaattaaggagatcaaagaaggctatctgagcctcgtgattcacgagataagtaagatggtaatcaaatacaacgcaattatcgccatggaagatctttcttatggttttaagaaaggccgcttcaaggtggagcggcaagtttaccagaaatttgaaaccatgctgattaataagttgaactatctggtcttcaaagacataagcatcacagagaatggagggctccttaagggctaccagctgacctatattccagataaattgaagaacgtgggtcatcaatgcggctgtatcttttacgtacccgctgcctatacttccaaaattgacccgaccacaggattcgtgaatatatttaagttcaaagatctgaccgttgacgcaaagcgcgaatttatcaaaaagttcgattcaattcggtacgacagcgagaaaaagctcttttgcttcacttttgattataacaatttcatcacccagaacacagtcatgagtaaatctagctggtccgtgtacacctatggggtacgcattaagcggcgctttgtgaatggccggttctcaaacgaaagcgacactatagatatcaccaaagacatggagaagacacttgaaatgaccgatattaattggcgcgacggtcacgatctgcggcaggacatcattgattacgagatagttcaacatatctttgaaattttccgcttgactgtccagatgcggaacagtctgtctgagctcgaagaccgcgattatgaccggcttatcagccctgtgctgaatgagaacaatattttttacgattccgccaaagctggcgacgccttgcccaaggatgcagacgccaacggagcttattgtatagccctgaaagggctctacgaaatcaagcagattaccgagaattggaaagaagatggcaagttctcacgcgacaaacttaagatcagcaacaaagattggtttgacttcattcaaaataagcggtatctgggcggctcccctgctgctaaacgtgttaagcttgatgggggtagcccggcagccaagagagtcaaactcgacggcggctccccagcggcaaaaagggtgaaactagacgggggtagccccgccgcgaagcgtgtaaagctggatggaggatcgcctgcggctaagcgagtcaaattagacggtgggtcaccggctgcaaagcgcgtcaaattggatgctagcggcagccaccaccaccaccaccactga

SEQ ID NO: 10 - DNA, 164 bp, Artificial Sequence
  insert1
gcgccaaattgattagtgacatcctgccggaattcgttattcacaataacaattactctgctagcgagaaggaagagaaaacccaagtcataaagctcttttcccggttcgccacttcatttaaagattatttccgcaaccgcgcaaattgctttagcgccgac

SEQ ID NO: 11 - DNA, 53 bp, Artificial Sequence
  insert1_primer_forward
gcgccaaattgattagtgacatcctgccggaattcgttattcacaataacaat

SEQ ID NO: 12 - DNA, 54 bp, Artificial Sequence
  insert1_primer_reverse
gtcggcgctaaagcaatttgcgcggttgcggaaataatctttaaatgaagtggc

SEQ ID NO: 13 - DNA, 1083 bp, Artificial Sequence
  insert2
cgcaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggcccgc

SEQ ID NO: 14 - DNA, 30 bp, Artificial Sequence
  insert2_primer_forward
cgcaaccgcgcaaattgctttagcgccgac

SEQ ID NO: 15 - DNA, 30 bp, Artificial Sequence
  insert2_primer_reverse
gcgggccagagtcggaatgccgaagttcaa

SEQ ID NO: 16 - DNA, 281 bp, Artificial Sequence
  insert3
ttgaacttcggcattccgactctggcccgcggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattct

SEQ ID NO: 17 - DNA, 50 bp, Artificial Sequence
  insert3_primer_forward
ttgaacttcggcattccgactctggcccgcggatggagcaagagtaaaga

SEQ ID NO: 18 - DNA, 50 bp, Artificial Sequence
  insert3_primer_reverse
agaatatatgcgctgggcttgtaggtctccactcctgtttttgaggagag

SEQ ID NO: 19 - protein, 1379 aa, Artificial Sequence
  opmgCas12a-1-6XNLS-6HIS
MGSSHHHHHHSSGLVPRGSHMASMTGGQQMGRGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFRNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGSPAAKRVKLDGGSPAAKRVKLDGGSPAAKRVKLDGGSPAAKRVKLDGGSPAAKRVKLDGGSPAAKRVKLDASGSHHHHHH

SEQ ID NO: 20 - DNA, 4140 bp, Artificial Sequence
  polynucleotides coding opmgCas12a-1-6XNLS-6HIS
atgggcagcagccatcatcatcatcatcacagcagcggcctggtgccgcgcggcagccatatggctagcatgactggtggacagcaaatgggtcgcggatccatgaacaatggcaccaacaatttccagaactttatcggaattagcagtctgcaaaagactctccggaatgcccttatacccaccgagacaacccagcagttcatcgtgaaaaacgggattatcaaggaagacgagctgcgcggcgaaaatcggcaaattttgaaagatataatggacgattattaccgcggttttatctctgagactctgagctccattgacgatatcgactggacctcactcttcgaaaagatggagattcagcttaaaaacggcgataataaggacacactgataaaagaacaggctgagaagcggaaagccatctataagaaatttgcagatgacgatcgcttcaagaacatgtttagcgccaaattgattagtgacatcctgccggaattcgttattcacaataacaattactctgctagcgagaaggaagagaaaacccaagtcataaagctcttttcccggttcgccacttcatttaaagattatttccgcaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggcccgcggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattcttgaagggtacaaacaaaacaagcatctgaaaagttctaaggactttgatatcactttctgtcacgacttgattgattattttaaaaattgcatagccatccatccggagtggaagaacttcggctttgacttcagcgatacctccacatacgaagacatttcaggtttttatcgcgaggttgaactgcagggctacaaaatcgattggacctatattagcgagaaggacatagatctccttcaggaaaaaggacaactgtacttgttccagatctataataaggactttagtaaaaagtctactgggaacgataatctgcacaccatgtacctcaaaaaccttttcagcgaggaaaatctgaaggacattgtcttgaaactgaacggcgaggctgaaatctttttccggaagtcctcaattaaaaatcctataatccataagaaaggtagcattctcgtgaaccgcacatatgaggccgaagagaaggatcagtttggcaatatccaaattgtacggaaaaccatacccgaaaacatctaccaggagctttataagtacttcaatgacaaaagtgataaggaactgtctgacgaggcagccaaattgaagaacgtggttggacaccatgaagctgccactaatattgtcaaagattatcgctacacctatgacaagtactttctgcacatgccgatcacaattaacttcaaagcaaataagaccagctttataaacgatcggattctccagtatattgccaaagagaagaatcttcatgtgatcgggattgaccgcggcgaacggaacctgatatacgtatccgtgatcgatacttgtggtaatattgttgagcaaaaatcattcaacatcgtcaatggctatgactaccagattaagttgaaacagcaagaaggagctcgccagatagcccggaaggagtggaaggaaatcgggaaaattaaggagatcaaagaaggctatctgagcctcgtgattcacgagataagtaagatggtaatcaaatacaacgcaattatcgccatggaagatctttcttatggttttaagaaaggccgcttcaaggtggagcggcaagtttaccagaaatttgaaaccatgctgattaataagttgaactatctggtcttcaaagacataagcatcacagagaatggagggctccttaagggctaccagctgacctatattccagataaattgaagaacgtgggtcatcaatgcggctgtatcttttacgtacccgctgcctatacttccaaaattgacccgaccacaggattcgtgaatatatttaagttcaaagatctgaccgttgacgcaaagcgcgaatttatcaaaaagttcgattcaattcggtacgacagcgagaaaaagctcttttgcttcacttttgattataacaatttcatcacccagaacacagtcatgagtaaatctagctggtccgtgtacacctatggggtacgcattaagcggcgctttgtgaatggccggttctcaaacgaaagcgacactatagatatcaccaaagacatggagaagacacttgaaatgaccgatattaattggcgcgacggtcacgatctgcggcaggacatcattgattacgagatagttcaacatatctttgaaattttccgcttgactgtccagatgcggaacagtctgtctgagctcgaagaccgcgattatgaccggcttatcagccctgtgctgaatgagaacaatattttttacgattccgccaaagctggcgacgccttgcccaaggatgcagacgccaacggagcttattgtatagccctgaaagggctctacgaaatcaagcagattaccgagaattggaaagaagatggcaagttctcacgcgacaaacttaagatcagcaacaaagattggtttgacttcattcaaaataagcggtatctgggcggctcccctgctgctaaacgtgttaagcttgatgggggtagcccggcagccaagagagtcaaactcgacggcggctccccagcggcaaaaagggtgaaactagacgggggtagccccgccgcgaagcgtgtaaagctggatggaggatcgcctgcggctaagcgagtcaaattagacggtgggtcaccggctgcaaagcgcgtcaaattggatgctagcggcagccaccaccaccaccaccactga

SEQ ID NO: 21 - DNA, 21 bp, Artificial Sequence
  HPRT1
ggttaaagatggttaaatgat

SEQ ID NO: 22 - protein, 1373 aa, Artificial Sequence
  opmgCas12a-1-6XNLS
MGSSHHHHHHSSGLVPRGSHMASMTGGQQMGRGSMNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFRNRANCFSADDISSSSCHRIVNDNAEIFFSNALVYRRIVKNLSNDDINKISGDIKDSLKEMSLEEIYSYEKYGEFITQEGISFYNDICGKVNSFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYTVISLYNLVRNYVTQKPYSTKKIKLNFGIPTLARGWSKSKEYSNNAIILMRDNLYYLGIFNAKNKPDKKIIEGNTSENKGDYKKMIYNLLPGPNKMIPKVFLSSKTGVETYKPSAYILEGYKQNKHLKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKTIPENIYQELYKYFNDKSDKELSDEAAKLKNVVGHHEAATNIVKDYRYTYDKYFLHMPITINFKANKTSFINDRILQYIAKEKNLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKKLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLGGSPAAKRVKLDGGSPAAKRVKLDGGSPAAKRVKLDGGSPAAKRVKLDGGSPAAKRVKLDGGSPAAKRVKLDASGS

SEQ ID NO: 23 - DNA, 4122 bp, Artificial Sequence
  polynucleotides coding opmgCas12a-1-6XNLS
atgggcagcagccatcatcatcatcatcacagcagcggcctggtgccgcgcggcagccatatggctagcatgactggtggacagcaaatgggtcgcggatccatgaacaatggcaccaacaatttccagaactttatcggaattagcagtctgcaaaagactctccggaatgcccttatacccaccgagacaacccagcagttcatcgtgaaaaacgggattatcaaggaagacgagctgcgcggcgaaaatcggcaaattttgaaagatataatggacgattattaccgcggttttatctctgagactctgagctccattgacgatatcgactggacctcactcttcgaaaagatggagattcagcttaaaaacggcgataataaggacacactgataaaagaacaggctgagaagcggaaagccatctataagaaatttgcagatgacgatcgcttcaagaacatgtttagcgccaaattgattagtgacatcctgccggaattcgttattcacaataacaattactctgctagcgagaaggaagagaaaacccaagtcataaagctcttttcccggttcgccacttcatttaaagattatttccgcaaccgcgcaaattgctttagcgccgacgatatcagttctagctcctgtcatcggattgtgaacgacaatgctgaaatcttcttttcaaacgcccttgtataccgccggattgtgaaaaatctgagcaacgatgacataaataagatcagtggagatattaaagactctttgaaggagatgagcctggaagagatctattcctacgaaaaatatggggagttcattacccaggaaggcatatcattttacaacgatatctgcggtaaggttaatagcttcatgaacctctattgtcagaaaaataaggagaacaaaaatctttacaagctgcgcaaattgcacaagcaaattctgtgcatcgcagacacaagttatgaagtcccttacaaatttgagtctgatgaagaggtgtatcagagcgtaaacggcttcctcgacaatatttcctcaaagcatatagtggaacggcttcgcaaaatcggagataactacaatgggtataacctggacaagatttacatcgttagcaaattttatgagagtgtctctcagaagacctaccgggattgggaaactattaataccgccttggagatacactataacaatatcctgcccggcaacggtaaaagcaaggctgacaaagtgaagaaagccgtaaagaatgatctccaaaaatccattacagaaatcaacgagcttgtgtcaaattacaagctgtgtccggacgataacattaaagcagaaacctatatacatgagatcagccacattttgaataacttcgaagcccaggagctgaagtacaatccagaaatccatctcgttgagagtgaacttaaagcttctgagctgaagaacgtcttggacgtgattatgaatgcctttcactggtgcagcgtattcatgactgaagagctggtggataaagacaacaatttttatgcagaactcgaggaaatatacgatgagatctataccgttatttccctttacaacctggtccgcaattatgtgacacagaagccctactcaaccaaaaagatcaaattgaacttcggcattccgactctggcccgcggatggagcaagagtaaagaatattctaataacgctataatcctcatgcgggataatctttactatctggggatttttaacgccaagaataaacctgacaagaaaatcattgagggcaacaccagcgaaaataagggtgattacaaaaagatgatatataacttgctgcccggcccgaataaaatgatcccaaaggtattcctctcctcaaaaacaggagtggagacctacaagcccagcgcatatattcttgaagggtacaaacaaaacaagcatctgaaaagttctaaggactttgatatcactttctgtcacgacttgattgattattttaaaaattgcatagccatccatccggagtggaagaacttcggctttgacttcagcgatacctccacatacgaagacatttcaggtttttatcgcgaggttgaactgcagggctacaaaatcgattggacctatattagcgagaaggacatagatctccttcaggaaaaaggacaactgtacttgttccagatctataataaggactttagtaaaaagtctactgggaacgataatctgcacaccatgtacctcaaaaaccttttcagcgaggaaaatctgaaggacattgtcttgaaactgaacggcgaggctgaaatctttttccggaagtcctcaattaaaaatcctataatccataagaaaggtagcattctcgtgaaccgcacatatgaggccgaagagaaggatcagtttggcaatatccaaattgtacggaaaaccatacccgaaaacatctaccaggagctttataagtacttcaatgacaaaagtgataaggaactgtctgacgaggcagccaaattgaagaacgtggttggacaccatgaagctgccactaatattgtcaaagattatcgctacacctatgacaagtactttctgcacatgccgatcacaattaacttcaaagcaaataagaccagctttataaacgatcggattctccagtatattgccaaagagaagaatcttcatgtgatcgggattgaccgcggcgaacggaacctgatatacgtatccgtgatcgatacttgtggtaatattgttgagcaaaaatcattcaacatcgtcaatggctatgactaccagattaagttgaaacagcaagaaggagctcgccagatagcccggaaggagtggaaggaaatcgggaaaattaaggagatcaaagaaggctatctgagcctcgtgattcacgagataagtaagatggtaatcaaatacaacgcaattatcgccatggaagatctttcttatggttttaagaaaggccgcttcaaggtggagcggcaagtttaccagaaatttgaaaccatgctgattaataagttgaactatctggtcttcaaagacataagcatcacagagaatggagggctccttaagggctaccagctgacctatattccagataaattgaagaacgtgggtcatcaatgcggctgtatcttttacgtacccgctgcctatacttccaaaattgacccgaccacaggattcgtgaatatatttaagttcaaagatctgaccgttgacgcaaagcgcgaatttatcaaaaagttcgattcaattcggtacgacagcgagaaaaagctcttttgcttcacttttgattataacaatttcatcacccagaacacagtcatgagtaaatctagctggtccgtgtacacctatggggtacgcattaagcggcgctttgtgaatggccggttctcaaacgaaagcgacactatagatatcaccaaagacatggagaagacacttgaaatgaccgatattaattggcgcgacggtcacgatctgcggcaggacatcattgattacgagatagttcaacatatctttgaaattttccgcttgactgtccagatgcggaacagtctgtctgagctcgaagaccgcgattatgaccggcttatcagccctgtgctgaatgagaacaatattttttacgattccgccaaagctggcgacgccttgcccaaggatgcagacgccaacggagcttattgtatagccctgaaagggctctacgaaatcaagcagattaccgagaattggaaagaagatggcaagttctcacgcgacaaacttaagatcagcaacaaagattggtttgacttcattcaaaataagcggtatctgggcggctcccctgctgctaaacgtgttaagcttgatgggggtagcccggcagccaagagagtcaaactcgacggcggctccccagcggcaaaaagggtgaaactagacgggggtagccccgccgcgaagcgtgtaaagctggatggaggatcgcctgcggctaagcgagtcaaattagacggtgggtcaccggctgcaaagcgcgtcaaattggatgctagcggcagctga

SEQ ID NO: 24 - DNA, 112 bp, Artificial Sequence
  Cpf1 enhancer 1
tggataataatgaacgcattagatagatttgaatgccggaactttggatttagatcacccattgacttggtcaacggaaacatgttcgctattaatatagcgaacatgtttc

SEQ ID NO: 25 - DNA, 112 bp, Artificial Sequence; identical to SEQ ID NO: 24
  Cpf1 enhancer 2